(19) United States
(12) Patent Application Publication
(10) Pub. No.: US 2020/0129044 A1
(43) Pub. Date: Apr. 30, 2020

(54) MEDICAL OBSERVATION APPARATUS AND MEDICAL OBSERVATION SYSTEM

(71) Applicant: Sony Olympus Medical Solutions Inc., Tokyo (JP)

(72) Inventor: Nobusuke YAMAOKA, Chiba (JP)

(73) Assignee: Sony Olympus Medical Solutions Inc., Tokyo (JP)

(21) Appl. No.: 16/660,834

(22) Filed: Oct. 23, 2019

(30) Foreign Application Priority Data
Oct. 30, 2018 (JP) 2018-203784

(51) Int. Cl. A61B 1/00; A61B 1/05; A61B 90/00

(52) U.S. Cl. CPC A61B 1/00013; A61B 1/05; A61B 90/25; A61B 90/361; A61B 2090/373; A61B 90/37

(57) ABSTRACT

A medical observation apparatus includes: an image sensor configured to image an observation target and generate a medical captured image by sequential reading; and a controller configured to control lighting of light sources configured to irradiate light to the observation target. The controller is configured to perform a first control including a control of lighting a light source configured to irradiate white light, and a second control including a control of lighting a light source configured to irradiate excitation light, and the first control is performed in a period that spans over a boundary of successive frames.

The present application claims priority to and incorporates by reference the entire contents of Japanese Patent Application No. 2018-203784 filed in Japan on Oct. 30, 2018.

### BACKGROUND

The present disclosure relates to a medical observation apparatus and a medical observation system.

There are cases where medical observation apparatuses that enable to observe an observation target, such as a part to be treated in an enlarged manner are used, for example, to support microsurgery such as neurosurgical operation, or to perform endoscopic surgery in medical sites. The medical observation apparatuses include a medical observation apparatus equipped with an optical microscope, and a medical observation apparatus equipped with an imaging device that has a function as an electronic imaging microscope. Hereinafter, the medical observation apparatus equipped with an optical microscope is referred to as “optical medical-observation apparatus”. Moreover, hereinafter, the medical observation apparatus equipped with an imaging device is referred to as “electronic-imaging medical-observation apparatus” or simply “medical observation apparatus” in some cases. Moreover, hereinafter, a captured image (moving image or still image, and the same applies hereafter) of an observation target imaged by an imaging device mounted on the medical observation apparatus is referred to as “medical captured image”.

With the improvement in image quality of imaging devices and the improvement in image quality of display devices on which a captured image is displayed, electronic-imaging medical-observation apparatuses have become possible to achieve the image quality equivalent to or higher than that of the optical medical-observation apparatuses. Furthermore, it is not necessary for a user (for example, a medical staff, such as an operator and an assistant of the operator, and the same applies hereafter) that uses the electronic-imaging medical-observation apparatus to look into an eyepiece constituting an optical microscope as in the case in which the optical medical-observation apparatus is used and, therefore, is possible to change the position of the imaging device flexibly. Thus, use of the electronic-imaging medical-observation apparatus has an advantage of being capable of supporting microsurgery more flexibly, and use of the electronic-imaging medical-observation apparatus in medical sites have been increasing.

In such a situation, a technique relating to an “imaging device that performs imaging while switching multiple illumination lights in a time division manner” has been developed. Examples of the above technique include the technique disclosed in International Publication Pamphlet No. WO 2013/099942.

According to one aspect of the present disclosure, there is provided a medical observation apparatus including: an image sensor configured to image an observation target and generate a medical captured image by sequential reading; and a controller configured to control lighting of light sources configured to irradiate light to the observation target, wherein the controller is configured to perform a first control including a control of lighting a light source configured to irradiate white light, and a second control including a control of lighting a light source configured to irradiate excitation light, and the first control is performed in a period that spans over a boundary of successive frames.

### SUMMARY

For example, when it is attempted to obtain an “image captured by irradiating white light to an observation target” and an “image captured by irradiating excitation light to an observation target” by an imaging device that images a medical captured image by the sequential reading by using an existing technique, such as the technique described in International Publication Pamphlet No. WO 2013/099942, the excitation light and the white light are alternately irradiated every two-frame period. One frame period according to the present embodiment is, for example, a “period from a point of time when exposure to light of the front line to be read by an image sensor in an imaging device is started until a point of time when exposure to light of the front line to be read again after all lines have been read is started”.

However, as in the case in which the existing technique is used, when the excitation light and the white light are irradiated alternately every two-frame period, a period in which exposure to the white light and exposure to the excitation light are mixed is generated. Moreover, a medical captured image obtained by imaging in the period in which the exposure to the white light and the exposure to the excitation light are mixed is an image not suitable for observation of a target to be observed by a medical staff, such as an operator. Therefore, when the existing technique is used, a medical captured image obtained by imaging in the period in which exposure to the white light and exposure to the excitation light are mixed is often not displayed on a display screen of a display device. “Not displaying a medical captured image obtained by imaging in a period in which exposure to the white light and exposure to the excitation light are mixed on a display screen of a display device” causes reduction of frame rate. As a result, it can make it difficult for an operator to perform procedures, and cause reduction in usability for a medical staff that uses the medical observation apparatus. Hereinafter, a person that uses the medical observation apparatus is referred to as “use of a medical observation apparatus”, or simply “user”.

There is a need for a new and improved medical observation apparatus and medical observation system that enables to improve usability for a user of the medical observation apparatus.

### BRIEF DESCRIPTION OF THE DRAWINGS

FIG. 1 is an explanatory diagram illustrating a first example of a configuration of a medical observation system according to a present embodiment;

FIG. 2 is an explanatory diagram illustrating an example of a use case in which the medical observation system according to the present embodiment is used;

FIG. 3 is an explanatory diagram for explaining an example of a configuration of an imaging device included in a medical observation apparatus according to the present embodiment;

FIG. 4 is an explanatory diagram illustrating a second example of the configuration of the medical observation system according to the present embodiment;

FIG. 5 is a functional block diagram illustrating an example of the configuration of the medical observation apparatus according to the present embodiment;

FIG. 6 is an explanatory diagram illustrating a first example of a timing of a first control according to a light-source control method according to the present embodiment;

FIG. 7 is an explanatory diagram illustrating an example of a medical captured image that is obtained when the first control is performed in the timing according to the first example in FIG. 6;

FIG. 8 is an explanatory diagram illustrating a second example of a timing of the first control according to the light-source control method according to the present embodiment;

FIG. 9 is an explanatory diagram for explaining an example of a medical captured image that is obtained when the first control is performed in the timing according to the second example in FIG. 8; FIG. 10 is an explanatory diagram illustrating third example of the first control timing according to the light source control method according to the present embodiment;

FIG. 11 is an explanatory diagram illustrating an example of a medical captured image that is obtained when the first control is performed in the timing according to the third example in FIG. 10;

FIG. 12 is an explanatory diagram illustrating an example of an imaging operation achieved by the first control and the second control according to the light source control method being performed in the medical observation apparatus illustrated in FIG. 5; and

FIG. 13 is a functional block diagram illustrating another example of the configuration of the medical observation apparatus according to the present embodiment.

### DETAILED DESCRIPTION

Hereinafter, exemplary embodiments of the present disclosure will be described in detail with reference to the accompanying drawings. Like reference symbols are assigned to like components having practically the same functional configurations throughout the present application and the drawings, and duplicated explanation is thereby omitted.

In the following, it will be described in the order below.
1. Medical Observation System According to Present Embodiment and Light-Source Control Method According to Present Embodiment

[1] Configuration of Medical Observation System

[1-1] Medical Observation System According to First Example

[1-2] Medical Observation Apparatus According to Second Example

[2] Functional Configuration of Medical Observation Apparatus According to Present Embodiment, and Processing Related to Light-Source Control Method

[3] One Example of Effect Produced by Using Light-Source Control Method According to Present Embodiment
2. Program According to Present Embodiment

Medical Observation System According to Present Embodiment and Light-Source Control Method According to Present Embodiment

Hereinafter, while describing an example of a medical observation system according to the present embodiment, a display control method according to the present embodiment will be described.

In the following, a case in which a medical observation apparatus according to the present embodiment performs processing of a light-source control method according to the present embodiment will be mainly described. In the medical observation system according to the present embodiment, an apparatus that can perform the processing of the light-source control method according to the present embodiment is not limited to the medical observation apparatus according to the present embodiment. For example, in the medical observation system according to the present embodiment, any apparatus, such as a medical controller, may perform the processing of the light-source control method according to the present embodiment.

[1] Configuration of Medical Observation System

[1-1] Medical Observation System According to First Example

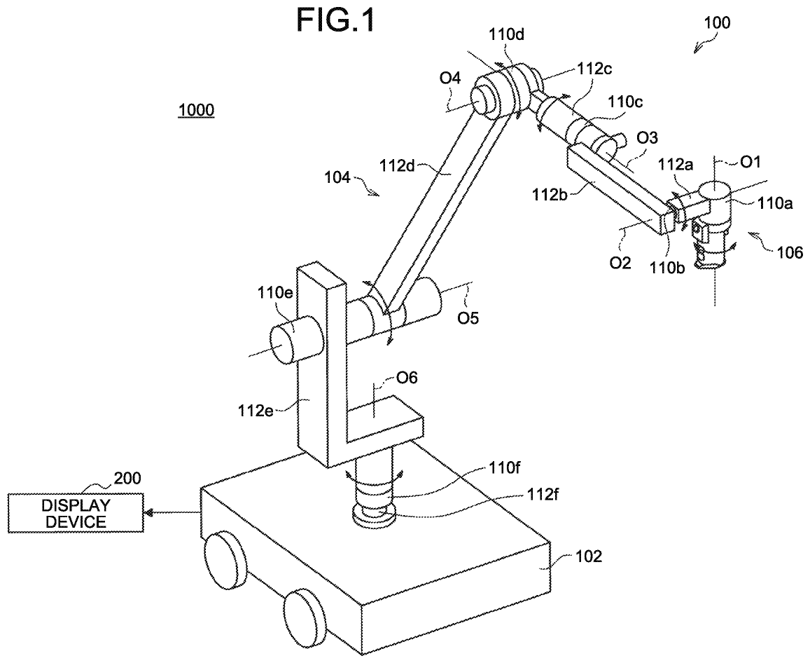

FIG. 1 is an explanatory diagram illustrating a first example of a configuration of a medical observation system 1000 according to the present embodiment. The medical observation system 1000 illustrated in FIG. 1 includes, for example, a medical observation apparatus 100 and a display device 200.

Note that the medical observation system according to the first example is not limited to the example illustrated in FIG. 1.

For example, the medical observation system according to the first example may further include a medical control apparatus (not shown) that controls various kinds of operations in the medical observation apparatus 100. The medical observation system 1000 illustrated in FIG. 1 shows

Attorney Docket No. 13346US01 an example in which, by providing a control unit (described later) in the medical observation apparatus 100, the medical observation apparatus 100 is given the function of the medical control apparatus (not shown) as described later.

Examples of the medical control apparatus (not shown) include a “medical controller”, a “computer such as a server”, and the like. Moreover, the medical control apparatus (not shown) may be an integrated circuit (IC) that can be incorporated in the device as above.

Furthermore, the medical observation system according to the first example may have a configuration including multiple units of one of or both of the medical observation apparatuses 100 and the display device 200. When multiple units of the medical observation apparatuses 100 are included, processing related to the light-source control method described later is performed in each of the medical observation apparatuses 100. Moreover, when the medical observation system according to the first example has a configuration including multiple units of the medical observation apparatuses 100 and the display devices 200, the medical observation apparatuses 100 and the display devices 200 may be associated with each other in one to one correspondence, or multiple units of the medical observation apparatuses 100 may be associated with a single unit of the display device 200. When multiple units of the medical observation apparatuses 100 are associated with a single unit of the display device 200, for example, in the display device 200, a switching operation is performed, thereby choosing a medical captured image captured by which one of the medical observation apparatuses 100 to be displayed on the display screen.

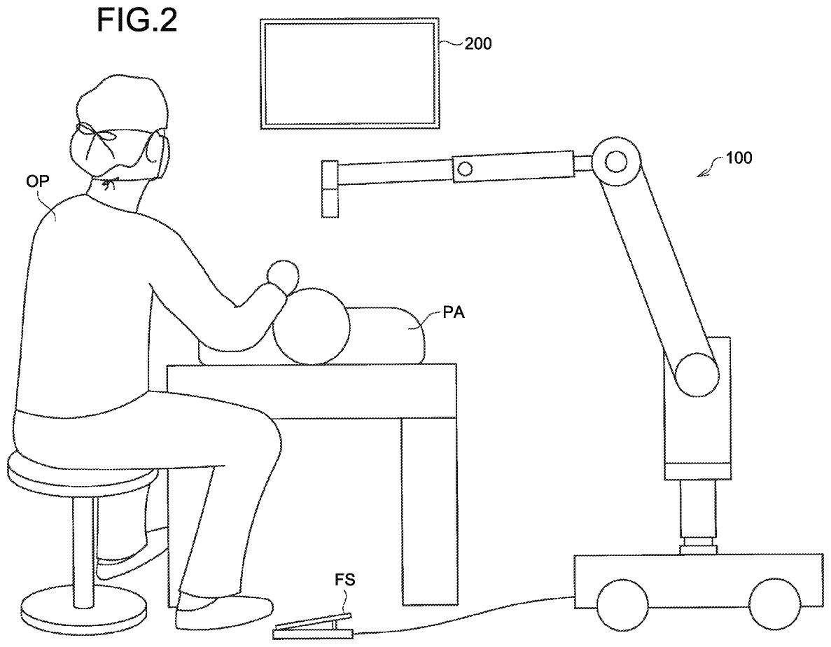

FIG. 2 is an explanatory diagram illustrating an example of a use case of the medical observation system 1000 according to the present embodiment, and illustrates an example of the use case in using the medical observation system 1000 according to the first example.

A patient PA (patient subject to medical treatment) being an observation target is imaged by an imaging device (described later) included in the medical observation apparatus 100. A captured image in which the patient PA subjected to the above medical treatment is imaged corresponds to an example of the medical captured image.

The medical captured image captured by the medical observation apparatus 100 is displayed on the display screen of the display device 200. An operator OP (an example of a user of the medical observation apparatus 100) that performs the medical treatment by using the medical observation apparatus 100 performs the medical treatment on the patient PA while viewing the medical captured image displayed on the display screen of the display device 200.

Moreover, the operator OP operates an external device of the medical observation apparatus 100, such as a foot switch FS, or an operating device (described alter) included in the medical observation apparatus 100, to move an arm (described later), an imaging device (described later), and the like included in the medical observation apparatus 100 and to bring the medical observation apparatus 100 to a desired condition.

Hereinafter, respective apparatuses constituting the medical observation system 1000 according to the first example illustrated in FIG. 1 will be described.

[1-1-1] Display Device 200

The display device 200 is a display unit in the medical observation system 1000 according to the first example, and corresponds to an external display device from the medical observation apparatus 100. The display device 200 displays various images, such as a medical captured image captured by the medical observation apparatus 100 and an image related to a user interface (UI), on the display screen. Moreover, the display device 200 may have a configuration enabling 3D display by an arbitrary method. Display in the display device 200 is controlled by, for example, the medical observation apparatus 100 or a medical control apparatus (not shown).

The display device 200 is arranged at an arbitrary position, such as on a wall, a ceiling, and a floor of an operating room, viewable for a person that is involved in an operation, such as an operator, in the operating room in the medical observation system 1000.

Examples of the display device 200 include a liquid crystal display, an organic electroluminescence (EL) display, a cathode ray tube (CRT) display, and the like.

Note that the display device 200 is not limited to the above examples. For example, the display device 200 may be an arbitrary wearable device that is used, being worn on operator's body, such as a head-mount display and an eyewear type display.

The display device 200 is actuated by power supplied by an internal power source, such as a battery provided in the display device 200, or by power supplied from an external power source connected thereto.

[1-1-2] Medical Observation Apparatus 100

The medical observation apparatus 100 illustrated in FIG. 1 is an example of the electronic-imaging medical-observation apparatus. For example, when the medical observation apparatus 100 illustrated in FIG. 1 is used at operation, an operator (an example of a user of the medical observation apparatus 100, and the same applies hereafter) observes a part to be treated, while viewing a medical captured image that is captured by the medical observation apparatus 100 and that is displayed on the display screen of the display device 200, and performs various kinds of treatment, such as procedure according to operation, with respect to the part to be treated.

As illustrated in FIG. 1, the medical observation apparatus 100 includes, for example, a base 102, an arm 104, and an imaging device 106.

Moreover, although not illustrated in FIG. 1, the medical observation apparatus 100 may include, for example, one or more processors (not shown) that is constituted of an arithmetic circuit, such as a micro processing unit (MPU), a read only memory (ROM) (not shown), a random access memory (RAM) (not shown), a recording medium (not shown), and a communication device (not shown). The medical observation apparatus 100 is actuated by power supplied from an internal power source, such as a battery provided in the medical observation apparatus 100, by power supplied from an external power source connected thereto, or the like.

The processor (not shown) functions as a control unit (described later) and a processing unit (described later) in the medical observation apparatus 100. The ROM (not shown) stores control data, such as a program and arithmetic parameters, used by the processor (not shown). The RAM (not shown) temporarily stores a program executed by the processor (not shown), and the like.

The recording medium (not shown) functions as a storage unit (not shown) in the medical observation apparatus 100. The recording medium (not shown) stores various kinds of data, such as data related to the light-source control method according to the present embodiment and various kinds of applications. Examples of the recording medium (not shown) include a magnetic recording medium such as a hard disk, a non-volatile memory such as a flash memory, and the like. Furthermore, the recording medium (not shown) may be detachable from the medical observation apparatus 100.

The communication device (not shown) is a communication unit included in the medical observation apparatus 100, and plays a role in communicating with an external device, such as the display device 200, in a wireless or wired manner. Examples of the communication device (not shown) include an IEEE 802.15.1 port and a transceiving circuit (wireless communication), an IEEE 802.11 port and a transceiving circuit (wireless communication), a communication antenna and a radio frequency (RF) circuit (wireless communication), a local area network (LAN) terminal and a transceiving circuit (wired communication), and the like.

[1-1-2-1] Base 102

The base 102 is a base of the medical observation apparatus 100, and one end of the arm 104 is connected thereto. The base 102 supports the arm 104 and the imaging device 106.

Moreover, for example, casters are provided in the base 102, and the medical observation apparatus 100 stands on the floor through the casters. By providing casters, the medical observation apparatus 100 is enabled to be moved easily on the floor with the casters.

[1-1-2-2] Arm 104

The arm 104 is structured with plural links connected with one another by joints.

The arm 104 supports the imaging device 106. The imaging device 106 supported by the arm 104 is three-dimensionally movable, and the imaging device 106 after a move is held by the arm 104 to maintain the position and the orientation.

More specifically, the arm 104 includes, for example, plural joints 110a, 110b, 110c, 110d, 110e, 110f, and plural links 112a, 112b, 112c, 112d, 112e, 112f that are rotatably joined with one another by the joints 110a, 110b, 110c, 110d, 110e, 110f. The rotatable range of the respective joints 110a, 110b, 110c, 110d, 110e, 110f are arbitrarily set to achieve desired movement of the arm 104 in a designing stage and a manufacturing stage.

[1-1-2-3] Imaging Device 106

The imaging device 106 images a medical captured image by rolling shutter method as the sequential reading. The imaging device 106 is supported by the arm 104, and images an observation target, such as a part to be treated of a patient. Imaging by the imaging device 106 is controlled by, for example, a processor that functions as a control unit described later, or an external medical control apparatus (not shown).

The imaging device 106 has a configuration corresponding to, for example, an electronic imaging microscope.

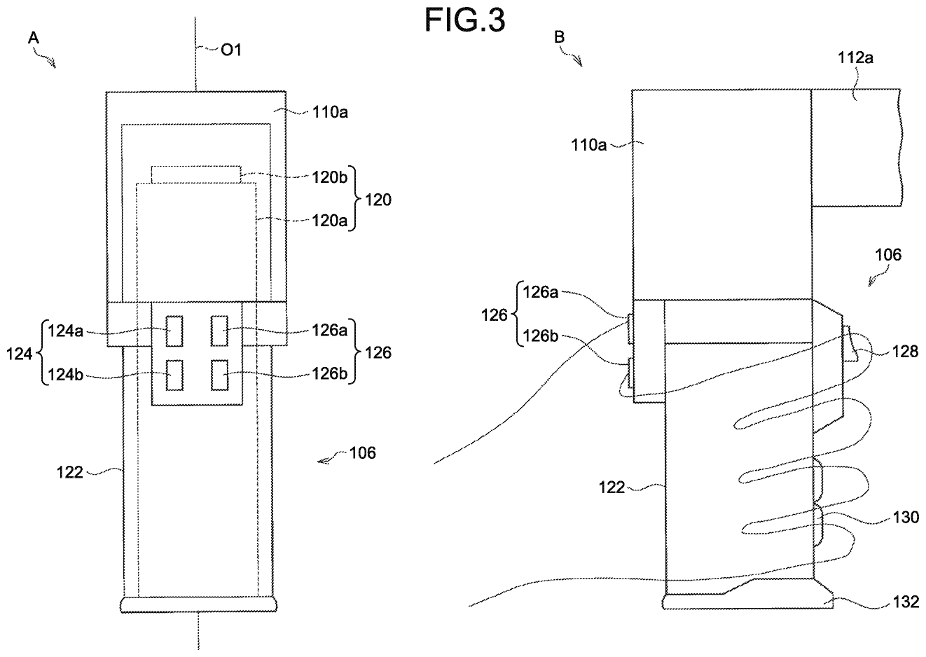

FIG. 3 is an explanatory diagram for explaining an example of a configuration of the imaging device 106 included in the medical observation apparatus 100 according to the present embodiment.

The imaging device 106 includes, for example, an imaging member 120, and a tubular member 122 having a substantially tubular shape. The imaging member 120 is arranged in the tubular member 122.

On an opening at a lower end (end on a lower side in FIG. 3) of the tubular member 122, for example, a cover glass (not shown) to protect the imaging member 120 is arranged.

Moreover, for example, a light source (not shown) is arranged inside the tubular member 122, and illumination light is irradiated to a subject through the cover glass from the light source at imaging. The light source provided inside the tubular member 122 includes, for example, a light source irradiating white light and a light source irradiating excitation light. The light sources irradiating white light and excitation light may be constituted of a single chip, or may be constituted of plural chips. Lighting of the light source arranged inside the tubular member 122 is controlled, for example, by a processor that functions as a control unit described later. Reflected light (observation light) from the subject on which the illumination light has been irradiated enters the imaging member 120 through the cover glass (not shown), an image signal expressing the subject (image signal expressing a medical captured image) is acquired by the imaging member 120.

As the imaging member 120, a configuration used in various kinds of publicly-known electronic imaging microscope unit can be applied.

As an example, the imaging member 120 is constituted of, for example, an optical system 120a, an image sensor 120b that includes an imaging device for imaging an image of an observation target by light that has passed through the optical system 120a. Moreover, the imaging member 120 further includes a filter that cuts off light of a wavelength corresponding to the excitation light from incident light. The filter is arranged on an optical path between the optical system 120a and the image sensor 120b.

By arranging the filter, entrance of light of the wavelength corresponding to the excitation light to the image sensor 120b is prevented. The optical system 120a is constituted of optical devices including, for example, one or more lenses, such as an objective lens, a zoom lens, and a focus lens, and a mirror. Examples of the image sensor 120b include, for example, an image sensor using multiple pixels (imaging devices), such as a complementary metal oxide semiconductor (CMOS).

The imaging member 120 may be configured to have two or more imaging devices constituted of the optical system 120a and the image sensor 120b, so as to function as so-called stereo camera. In the configuration of the imaging device 106 functioning as a stereo camera, the optical system may be a Galileo optical system, or a Greenough optical system.

The imaging member 120 has one or more functions that are generally equipped with in an electronic imaging microscope, such as a zoom function (one or both of an optical zoom function and an electronic zoom function) and an auto focus (AF) function.

Moreover, the imaging member 120 may be configured to enable so-called high resolution imaging, such as 4K and 8K. By configuring the imaging member 120 to be able to perform high resolution imaging, it becomes possible to display an image on the display device 200 having a large display screen of, for example, 50 inches or larger, while keeping a certain resolution (for example, Full HD quality, or the like). Therefore, the visibility for an operator that views the display screen improves. Furthermore, by configuring the imaging member 120 to be able to perform high resolution imaging, it becomes possible to keep a certain resolution even when a captured image is enlarged to be displayed on the display screen of the display device by an electronic zoom function. Moreover, when a certain resolution is kept even when an electronic zoom function is used, it is possible to reduce the performance capabilities of the optical zoom function in the imaging device 106 and, therefore, is possible to simplify the optical system of the imaging device 106, to structure the imaging device 106 in a smaller size.

In the imaging device 106, for example, various kinds of operating devices to control operation of the imaging device 106 are arranged. For example, in the example in FIG. 3, a zoom switch 124, a focus switch 126, and an operation-mode changing switch 128 are provided in the imaging device 106. It is needless to say that positions and forms in which the zoom switch 124, the focus switch 126, and the operation-mode changing switch 128 are arranged are not limited to the example illustrated in FIG. 3.

The zoom switch 124 and the focus switch 126 are an example of the operating device to adjust an imaging condition in the imaging device 106.

The zoom switch 124 includes, for example, a zoom-in switch 124a that increases a zoom factor (magnification), and a zoom-out switch 124b that reduces a zoom factor. When the zoom switch 124 is operated, the zoom factor is adjusted, and zoom is adjusted.

The focus switch 126 includes, for example, a wide-range focus switch 126a by which a focal length to an observation target (subject) is increased, and a close-range focus switch 126b by which a focal length to an observation target is decreased. By operating the focus switch 126, the focal length is adjusted, and the focus is adjusted.

The operation-mode changing switch 128 is an example of an operating device to change the operation mode of the arm 104 in the imaging device 106. By operating the operation-mode changing switch 128, the operation mode of the arm 104 is changed. The operation mode of the arm 104 includes, for example, the fixed mode and the free mode as described above.

An example of operation with respect to the operation-mode changing switch 128 includes operation of pressing the operation-mode changing switch 128. For example, during an operator presses the operation-mode changing switch 128, the operation mode of the arm 104 is in the free mode, and when the operation is not pressing the operation-mode changing switch 128, the operation mode of the arm 104 is in the fixed mode.

Furthermore, to improve the operability and the convenience at the time when an operator that performs operation with respect to the respective operating devices performs operation, for example, a non-slip member 130 and a projecting member 132 are arranged in the imaging device 106.

The non-slip member 130 is a member arranged to avoid slipping of an operating body, for example, at the time when the operator operates the tubular member 122 with the operating body, such as a hand. For example, the non-slip member 130 is made from a material having a high coefficient of friction, and has a less slippery structure, such as asperities.

The projecting member 132 is a member arranged to prevent the field of view of the optical system 120a from being blocked, for example, when the operator operates the tubular member 122 with an operating body, such as a hand, or to prevent the cover glass (not shown) from getting dirty by the operating body touching the cover glass when operation is performed with the operating body.

It is needless to say that the position and a form in which the non-slip member 130 and the projecting member 132 are arranged are not limited to the example illustrated in FIG. 3. Moreover, in the imaging device 106, one or both of the non-slip member 130 and the projecting member 132 may be absent.

Furthermore, the imaging device 106 has a configuration capable of switching among multiple observation modes. The observation modes according to the present embodiment include, for example, an observation mode in which imaging is performed with white light, an observation mode in which imaging is performed with excitation light, an observation mode in which imaging is performed by using an image enhancement observation technique, and the like. Hereinafter, observation by the observation mode in which imaging is performed with white light may be referred to as “white light observation”, and a medical captured image acquired by the white light observation may be referred to as “white-light captured image”, or “white light image”. Moreover, hereinafter, observation by an observation mode in which imaging is performed with fluorescence is referred to as “fluorescence observation”, and a medical captured image acquired by the fluorescence observation may be referred to as “fluorescence captured image” or “fluorescence image”.

The excitation light according to the present embodiment is, for example, light in a specific wavelength range that can make a fluorescent reagent, such as light of a near infrared wavelength range, light of a fluorescence wavelength range in the fluorescence observation using 5-aminolevulinic acid (5-ALA), and light of a fluorescence wavelength range of the fluorescence observation using indocyanine green. The excitation light or the fluorescence are called special light in some cases.

An example of configuration of the imaging device 106 enabling to switch among the observation modes includes, for example, a “configuration having a filter that passes light of a specific wavelength range, and that does not pass light of other wavelength range, and a moving mechanism that selectively arranges the filter on an optical path”. Examples of the specific wavelength range to be passed through the filter according to the present embodiment include a near infrared wavelength range (for example, a wavelength range of approximately 0.7 micrometers to 2.5 micrometers), a fluorescence wavelength range by the fluorescence observation using 5-ALA (for example, a wavelength range of approximately 0.6 micrometers to 0.65 micrometers), a fluorescence wavelength range by the fluorescence observation using indocyanine green (for example, a wavelength range of excitation light: 700 to 850 nanometers, fluorescence wavelength: 780 to 950 nanometers), and the like.

Note that multiple filters that pass light of different wavelength range may be provided in the imaging device 106. Moreover, the example in which imaging is performed with light of a specific wavelength range by arranging a filter on an optical path has been described in the above, but it is needless to say that the configuration of the imaging device 106 to perform imaging with light of a specific wavelength range are not limited to the example described above.

An image signal (image data) that is generated by imaging by the imaging device 106 is subjected to image processing, for example, by a processor functioning as a control unit described later. The image processing according to the present embodiment includes, for example, one or more kinds of processing out of various kinds of processing, such as gamma correction, white balance adjustment, enlargement and reduction by the electronic zoom function, inter-pixel correction, and synthesizing processing.

Note that when the medical observation system according to the present embodiment includes a medical control apparatus (not shown) that controls various kinds of operations in the medical observation apparatus 100, the image processing according to the present embodiment may be performed in the medical control apparatus (not shown).

The medical observation apparatus 100 transmits, for example, a display control signal and an image signal subjected to the image processing as described above to the display device 200.

As the display control signal and the image signal are transmitted to the display device 200, the a medical captured image in which an observation target is imaged (for example, a captured image in which a part to be treated is imaged) is displayed on the display screen of the display device 200. At this time, the medical captured image in which the observation target is imaged may be enlarged or reduced to be displayed by a desirable scaling factor by one of or both of the optical zoom function and the electronic zoom function, on the display screen of the display device 200.

The medical observation apparatus 100 illustrated in FIG. 1 has, for example, a hardware configuration described, referring to FIG. 1 and FIG. 3.

The hardware configuration of the medical observation apparatus according to the present embodiment is not limited to the configuration described referring to FIG. 1 and FIG. 3.

For example, the medical observation apparatus according to the present embodiment may have a configuration in which the arm 104 is directly attached to a ceiling or a wall of an operation room, without the base 102. For example, when the arm 104 is attached to a ceiling, the medical observation apparatus according to the present embodiment has a configuration in which the arm 104 is hang from the ceiling.

Moreover, the example in which the arm 104 is configured to realize six degrees of freedom in driving of the imaging device 106 is illustrated in FIG. 1, but the configuration of the arm 104 is not limited to the configuration with which the imaging device 106 has six degrees of freedom in driving. For example, as long as the arm 104 is configured to be able to move the imaging device 106 appropriately according to a use, the number and arrangement of joints and links, the direction of driving axes of the joints, and the like can be set appropriately to give desired freedom to the arm 104.

Furthermore, the example in which various kinds of operating devices to control operation of the imaging device 106 are provided in the imaging device 106 is illustrated in FIG. 1 and FIG. 3, but some of or all of the operating devices illustrated in FIG. 1 and FIG. 3 may be excluded to be provided in the imaging device 106. As an example, various kinds of operating devices to control operation of the imaging device 106 may be arranged in a part other than the imaging device 106 constituting the medical observation apparatus according to the present embodiment, as another example, various kinds of respective operating devices to control operation of the imaging device 106 may be an external operating device, such as a foot switch FS and a remote controller.

[1-2] Medical Observation System According to Second Example

The medical observation system 1000 according to the present embodiment is not limited to be configured as in the first example illustrated in FIG. 1. Next, as another example of the medical observation system 1000, an example of a configuration of the medical observation system 1000 that includes the medical observation apparatus 100 that functions as an endoscope device will be described.

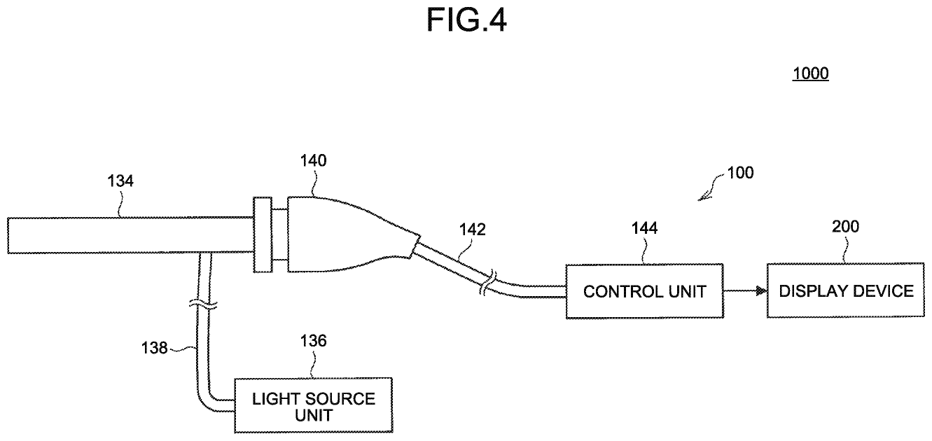

FIG. 4 is an explanatory diagram illustrating a second example of the configuration of the medical observation system 1000 according to the present embodiment. The medical observation system 1000 illustrated in FIG. 4 includes, for example, the medical observation apparatus 100 and the display device 200. For example, when the medical observation apparatus 100 illustrated in FIG. 4 is used at operation, an operator observes a part to be treated, while viewing a medical captured image that is captured by the medical observation apparatus 100 and that is displayed on the display screen of the display device 200, and performs various kinds of treatment, such as procedure according to operation, with respect to the part to be treated.

Note that the medical observation system according to the second example is not limited to the example in FIG. 4.

For example, the medical observation system according to the second example may further include a medical control apparatus (not shown) that controls various kinds of operations in the medical observation apparatus 100, similarly to the medical observation system according to the first example.

Furthermore, the medical observation system according to the second example may have a configuration including multiple units of one of or both of the medical observation apparatuses 100 and the display device 200, similarly to the medical observation system according to the first example.

Hereinafter, respective devices that constitute the medical observation system 1000 according to the second example illustrated in FIG. 4 will be described.

[1-2-1] Display Device 200

The display device 200 is a display unit in the medical observation system 1000 according to the second example, and corresponds to an external display device from the medical observation apparatus 100. The display device 200 constituting the medical observation system 1000 according to the second example is the same as the display device 200 constituting the medical observation system 1000 according to the first example.

[1-2-2] Medical Observation Apparatus 100

The medical observation apparatus 100 illustrated in FIG. 4 includes, for example, an inserting member 134, a light source unit 136, a light guide 138, a camera head 140, a cable 142, and a control unit 144. The medical observation apparatus 100 is actuated by power supplied from an internal power source, such as a battery provided in the medical observation apparatus 100, by power supplied from an external power source connected thereto, or the like.

The inserting member 134 has a long a slender shape, and has an optical system that gathers incident light, thereinside. A distal end of the inserting member 134 is inserted into, fore example, a body cavity of a patient. A proximal end of the inserting member 134 is detachably connected to a distal end of the camera head 140. Moreover, the inserting member 134 is connected to the light source unit 136 through the light guide 138, to be provided with light from the light source unit 136.

The inserting member 134 may be made from a material without flexibility, or may be made from a material having flexibility. According to the material forming the inserting member 134, the medical observation apparatus 100 can be called rigid endoscope or flexible endoscope.

The light source unit 136 is connected to the inserting member 134 through the light guide 138. The light source unit 136 provides light to the inserting member 134 through the light guide 138.

The light source unit 136 includes a light source irradiating white light and a light source irradiating excitation light. The light sources irradiating white light and excitation light may be constituted of a single chip, or may be constituted of plural chips. It is needless to say that an example of the light sources included in the light source unit 136 is not limited to the example described above.

The light source unit 136 is connected with the control unit 144 by wired or wireless connection, and lighting of the light sources in the light source unit 136 is controlled, for example, by the control unit 144 functioning as a control unit described later.

Light provided to the inserting member 134 is emitted from the distal end of the inserting member 134, and is irradiated to the observation target, such as a tissue inside a body cavity of a patient. Reflected light from the observation target is gathered by the optical system inside the inserting member 134.

The camera head 140 has a function of imaging an observation target. The camera head 140 is connected to the control unit 144 through the cable 142 that is a signal transmitting member.

The camera head 140 has an image sensor, and images the observation target by subjecting reflected light from the observation target gathered by the inserting member 134 to photoelectric conversion, and outputs an image signal (signal expressing a medical captured image) acquired by imaging to the control unit 144 through the cable 142. Examples of the image sensor in the camera head 140 include, for example, an image sensor using multiple imaging devices, such as a CMOS and a charge coupled device (CCD). The camera head 140 further includes a filter that cuts off light of a wavelength corresponding to the excitation light. The above filter is arranged on an optical path between the inserting member 134 and the image sensor. By arranging the filter, entrance of light of the wavelength corresponding to the excitation light to the image sensor is prevented.

In the medical observation apparatus 100 functioning as an endoscope device, for example, the inserting member 134 and the camera head 140 play a role as an “imaging device that is inserted into a body of a subject and that images an inside of the body”. Note that in the medical observation apparatus 100 functioning as an endoscope device, the inserting member 134, the light source unit 136, and the camera head 140 may play a role as the “imaging device that is inserted into a body of a subject and that images an inside of the body”.

The medical observation apparatus 100 functioning as an endoscope device may include, for example, plural imaging devices that function as a so-called stereo camera. In a configuration of the imaging devices that function as a stereo camera, the optical system may be a Galileo optical system or a Greenough optical system, similarly to the medical observation apparatus 100 constituting the medical observation system according to the first example.

The control unit 144 controls the imaging device. More specifically, the control unit 144 controls the respective components of the light source unit 136 and the camera head 140.

Moreover, the control unit 144 includes a communication device (not shown), and transmits an image signal output from the camera head 140 to the display device 200 by an arbitrary wireless communication or an arbitrary wired communication. The control unit 144 may transmit an image signal and a display control signal to the display device 200.

Examples of the communication device (not shown) included in the control unit 144 include an IEEE 802.15.1 port and a transceiving circuit (wireless communication), an IEEE 802.11 port and a transceiving circuit (wireless communication), a communication antenna and a radio frequency (RF) circuit (wireless communication), an optical communication device (wired communication or wireless communication), a local area network (LAN) terminal and a transceiving circuit (wired communication), and the like. The communication device (not shown) may be configured to be able to communicate with one, or with two or more external devices by multiple communication methods.

Furthermore, the control unit 144 may subject the image signal output from the camera head 140 to predetermined processing, and transmit the image signal subjected to the predetermined processing to the display device 200. The predetermined processing performed with respect to the image signal includes, for example, one or more kinds of processing out of various kinds of processing, such as gamma correction, white balance adjustment, enlargement and reduction by the electronic zoom function, inter-pixel correction, and synthesizing processing.

The control unit 144 may store a medical captured image based on the image signal.

As the control unit 144, for example, a camera control unit (CCU) may be applied.

The medical observation apparatus 100 functioning as an endoscope device has, for example, a hardware configuration described, referring to FIG. 4. In the medical observation apparatus 100 functioning as an endoscope, for example, the inserting member 134 and the camera head 140 play a role of an imaging device, and imaging by the imaging device is controlled by the control unit 144. Moreover, in the medical observation apparatus 100 functioning as an endoscope, for example, lighting of the light source in the light source unit 136 is controlled by the control unit 144.

[2] Functional Configuration of Medical Observation Apparatus According to Present Embodiment, and Processing Related to Light-Source Control Method

Next, an example of processing related to the light source control method according to the present embodiment will be described, while describing an example of the functional configuration of the medical observation apparatus 100 illustrated in FIG. 1 and FIG. 4. Hereinafter, a case in which the fluorescent reagent used in fluorescence observation is indocyanine green will be described. Note that the fluorescent reagent used in fluorescent observation is not limited to indocyanine green, but it may be any reagent as long as it is suitable for excitation light.

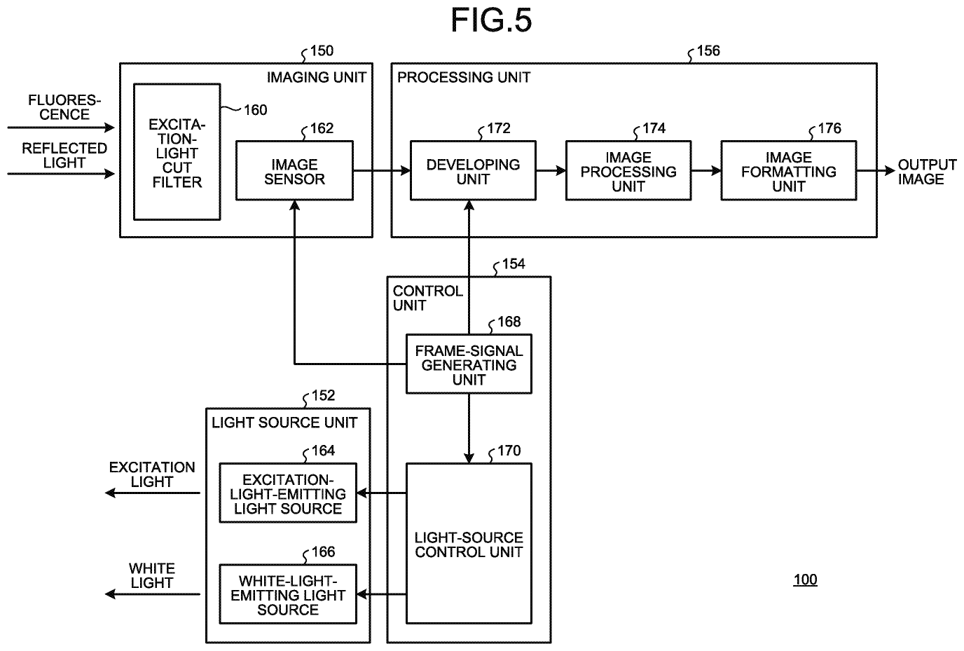

FIG. 5 is a functional block diagram illustrating an example of the configuration of the medical observation apparatus 100 according to the present embodiment. The medical observation apparatus 100 includes, for example, an imaging unit 150, a light source unit 152, a control unit 154, and a processing unit 156.

The imaging unit 150 includes an imaging device that images a medical captured image by rolling shutter as a sequential reading method. As illustrated in FIG. 5, when reflected light that is reflected from an observation target when white light is irradiated to the observation target, and fluorescence that is emitted by a fluorescence reagent present in the observation target when excitation light is irradiated to the observation target can enter the imaging unit 150. The imaging unit 150 is constituted of, for example, the “imaging device 106” (in the case of the medical observation apparatus 100 illustrated in FIG. 1), the “inserting member 134”, and the “camera head 140” (in the case of the medical observation apparatus 100 illustrated in FIG. 1). FIG. 5 illustrates an excitation-light cut filter 160 (filter that cuts off light of a wavelength corresponding to the excitation light) and an image sensor 162, as the imaging unit 150 for convenience sake. The image sensor 162 subjects incident light to photoelectric conversion. Signals subjected to photoelectric conversion by rolling shutter are sequentially read from a front line. The image sensor 162 is sensitive to both white light and excitation light. The excitation-light cut filter 160 may be a detachable filter, or may be a fixed filter. The imaging unit 150 may have a configuration without the excitation-light cut filter 160.

Imaging in the imaging unit 150 is controlled by, for example, the control unit 154.

The light source unit 152 includes an excitation-light-emitting light source 164 (an example of the light source that irradiates excitation light, and the same applies hereafter) and a white-light-emitting light source 166 (an example of the light source that irradiates white light, and the same applies hereafter). Lighting of the respective light sources constituting the light source unit 152 is controlled, for example, by processing related to the light-source control method by the control unit 154.

The control unit 154 is constituted of, for example, the processor (not shown) described above, and plays a role in controlling the entire medical observation apparatus 100.

Furthermore, the control unit 154 controls lighting of the light source included in the light source unit 152, that is, lighting of the light source that irradiates light to an observation target, by performing processing related to the light-source control method. The processing related to the light-source control method in the control unit 154 may be performed in a distributed manner in plural processing circuits (for example, plural processors, and the like).

The control unit 154 includes, for example, a frame-signal generating unit 168 and a light-source control unit 170.

The frame-signal generating unit 168 generates a signal (hereinafter, “frame signal”) indicating a start of a frame period, and transfers the frame signal to the imaging unit 150, the processing unit 156, and the light-source control unit 170. The frame-signal generating unit 168 transfers the frame signal to respective components according to a frame rate set to, for example, 120 [fps], or the like. The frame signal may be transferred to the imaging unit 150 through the processing unit 156. The frame signal corresponds to a signal to be respective triggers of imaging in the imaging unit 150, processing in the processing unit 156, and the processing in the light-source control unit 170.

The light-source control unit 170 performs processing related to the light-source control method based on the frame signal transferred from the frame-signal generating unit 168, and controls lighting of the respective excitation-light-emitting light source 164 and white-light-emitting light source 166 constituting the light source unit 152. That is, the processing related to the light-source control method in the control unit 154 is performed by the light-source control unit 170.

The light-source control unit 170 performs a first control including control of lighting the white-light-emitting light source 166, and a second control of lighting the excitation-light-emitting light source 164. As in an example described later, the first control is performed in a period over successive frames. Moreover, the first control is performed for a period shorter than the one frame period, and the second control is performed for a period longer than the period in which the first control is performed.

Specifically, the light-source control unit 170 repeats the first control, and performs the second control in a period in which at least the first control is not performed.

Examples of a case in which the second control is performed in a period in which at least the first control is not performed include, for example, examples described below.

The light-source control unit 170 preforms the second control in a period in which the first control is performed, and in a period in which the first control is not performed. In this case, the second control is performed all the time except when a third control described later is performed.

The light-source control unit 170 performs the second control in a period in which the first control is not performed, and performs the second control in a period in which the first control is being performed. In this case, the first control and the second control are performed alternately except when the third control described later is performed.

When the first control is repeated, and when a shutter speed in the imaging device constituting the imaging unit 150 is one frame period, the light-source control unit 170 performs the first control, for example, every two frame period. When the first control according to the light-source control method is repeated, the first control is not necessarily performed every two frame period. The first control can be performed, fore example, every period corresponding to a shutter speed in the imaging device constituting the imaging unit 150.

Furthermore, when the first control is repeated, the light-source control unit 170 performs the first control in predetermined timing that has been set. As an example of the predetermined timing, for example, timings in (A) to (C) described below are considered.

(A) First Example of Predetermined Timing

A predetermined timing in which the first control is performed is a timing in which lighting of the light source that irradiates white light spans over an end of reading a final line of a frame that is being read in the imaging device and a start of reading a front line of a frame to be read next.

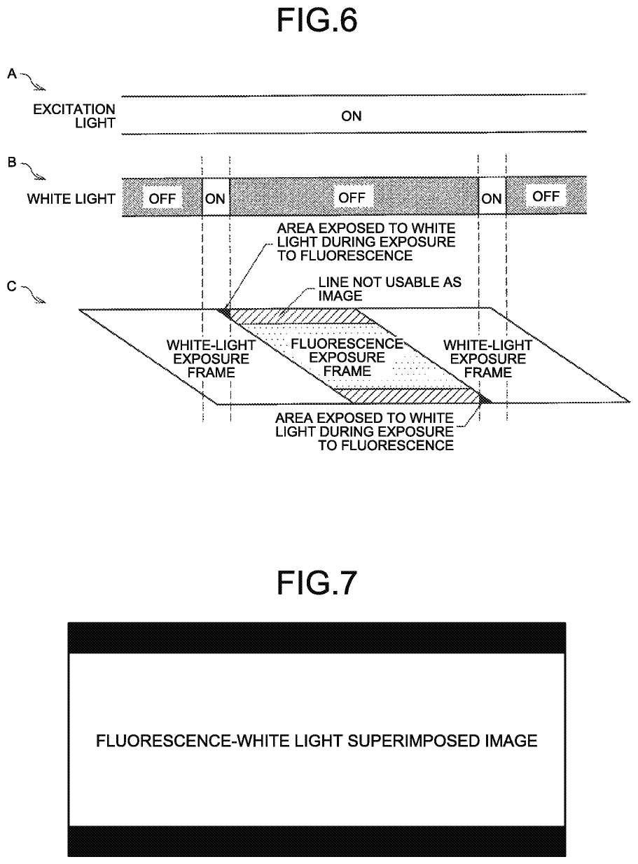

FIG. 6 is an explanatory diagram illustrating a first example of a timing of the first control according to the light-source control method according to the present embodiment. A in FIG. 6 illustrates an ON state of the light source that irradiates excitation light, and illustrates an example in which the excitation light is irradiated all the time. The medical observation apparatus 100 may turn off the light source that irradiates excitation light when the light source that irradiates white light is ON. B in FIG. 6 illustrates an example of an ON state of the light source that irradiates white light, and illustrates a first example of the first control timing. C in FIG. 6 illustrates an example of frames that are being read by the imaging device. FIG. 7 is an explanatory diagram illustrating an example of a medical captured image that is obtained when the first control is performed in the timing according to the first example in FIG. 6. The medical captured image illustrated in FIG. 7 is an “example in which a fluorescence captured image (an example of the medical captured image, and the same applies hereafter) obtained by the fluorescence observation and a white-light captured image (another example of the medical captured image, and the same applies hereafter) obtained by the white light observation are synthesized” by processing in the processing unit 156 described later. In FIG. 7, the synthesized image is referred to as “superimposed image” (hereinafter, other images are also the same). As described later, a medical captured image acquired by processing in the processing unit 156 is not limited to a synthesized image.

When the first control is performed in the “timing in which lighting of the light source that irradiates white light spans over an end of reading a final line of a frame that is being read in the imaging device and a start of reading a front line of a frame to be read next”, time of being exposed to white light is present in a fluorescence exposure frame as shown in portions filled in with black in C in FIG. 6. Because white light has larger light amount than fluorescence, a line overlapping the black portion in C in FIG. 6 cannot be used in a fluorescence captured image.

Accordingly, in the medical observation apparatus 100, a portion that is not usable as a fluorescence captured image in a medical observation image (a line portion overlapping the black portion in C in FIG. 6) is masked, not to show the portion on the display screen of the display device 200. The masking with respect to the medical observation image may be performed in the imaging unit 150, or may be performed in the processing unit 156. A portion in black in FIG. 7 is an example of a masked portion with respect to the medical observation image.

Even when a masked portion is present in a medical observation image as illustrated in FIG. 7, the operator can display a medical captured image in which a range of an observation target necessary for performing procedures is imaged, on the display screen of the display device 200 by one of or both of a zoom operation in the imaging device, and an adjustment of an observation view range by an operation of the arm 104. Therefore, even when a masked portion is present in a medical observation image as illustrated in FIG. 7, there is a low possibility that medical treatment by a medical staff is interfered.

Moreover, when the first control is performed in the “timing that spans over an end of reading a final line of a frame that is being read in the imaging device and a start of reading a front line of a frame to be read next”, ON time of the light source that irradiates white light is shorter than one frame period as illustrated in B in FIG. 6.

Thus, as the ON time of the light source that irradiates white light is set to be shorter than one frame period, the light-source control unit 170 increases one of or both of a light amount of the light source that irradiates white light and a gain of a signal indicating a medical captured image, accordingly. The gain of the signal indicating a medical captured image can be changed, for example, by an amplifier that is provided in one or both of the imaging unit 150 and the processing unit 156. As one example, when the ON time of the light source that irradiates white light is 1/5 frame period, the light-source control unit 170 increases the light amount of the light source that irradiates white light by five times. It is needless to say that the example of a control in the light-source control unit 170 is not limited to the example described above.

(B) Second Example of Predetermined Timing

The predetermined timing in which the first control is performed in a timing in which lighting of the light source that irradiates white light ends at the same time as an end of reading a final line of a frame that is being read by the imaging device.

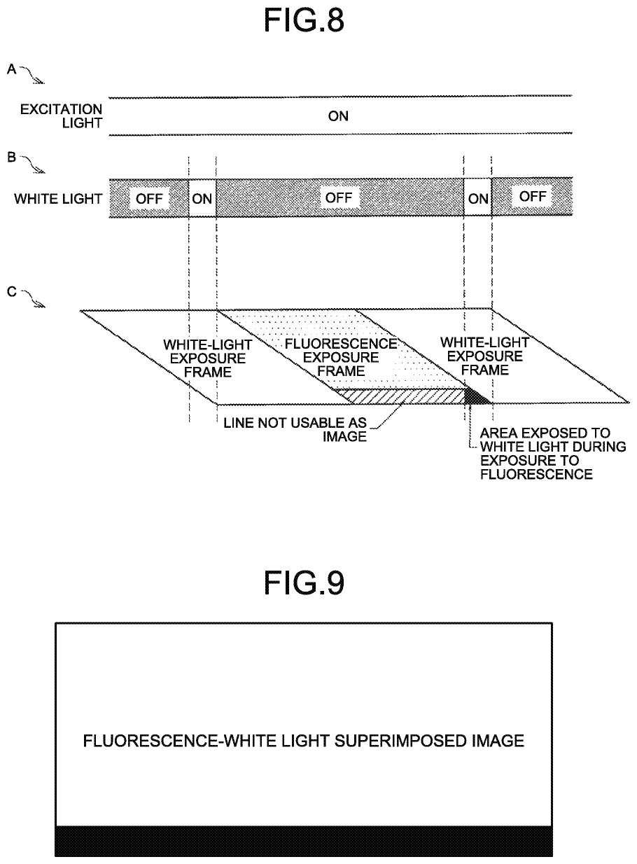

FIG. 8 is an explanatory diagram illustrating a second example of a timing of the first control according to the light-source control method according to the present embodiment. A in FIG. 8 illustrates an example of an ON state of the light source that irradiates excitation light, and illustrates an example in which excitation light is irradiated all the time. Note that the medical observation apparatus 100 may turn off the light source that irradiates excitation light during when the light source that irradiates white light is ON. B in FIG. 8 illustrates an example of an ON state of the light source that irradiates white light, and illustrates the second example of a timing of the first control. C in FIG. 8 illustrates one example of a frame that is being read by the imaging device. FIG. 9 is an explanatory diagram for explaining an example of a medical captured image that is obtained when the first control is performed in the timing according to the second example in FIG. 8. The medical captured image illustrated in FIG. 9 is, similarly to FIG. 7, the “example in which a fluorescence captured image obtained by the fluorescence observation and a white-light captured image obtained by the white light observation are synthesized”.

When the first control is performed in the “timing in which the first control is performed in a timing in which lighting of the light source that irradiates white light ends at the same time as an end of reading a final line of a frame that is being read by the imaging device”, time of being exposed to white light is present in a fluorescence exposure frame as it is filled in with black in C in FIG. 8.

Thus, in the medical observation apparatus 100, similarly to the case in which the first control is performed in the timing according to the first example indicated in (A) described above, a portion that is not usable as a fluorescence captured image in a medical observation image (a line portion overlapping the black portion in C in FIG. 8) is masked, not to show the portion on the display screen of the display device 200. A portion in black in FIG. 9 is an example of a masked portion with respect to the medical observation image.

Even when a masked portion is present in a medical observation image as illustrated in FIG. 9, the operator can display a medical captured image in which a range of an observation target necessary for performing procedures is imaged, on the display screen of the display device 200 by one of or both of a zoom operation in the imaging device, and an adjustment of an observation view range by an operation of the arm 104. Therefore, even when a masked portion is present in a medical observation image as illustrated in FIG. 9, there is a low possibility that medical treatment by a medical staff is interfered.

Moreover, when the first control is performed in the “timing in which lighting of the light source that irradiates white light ends at the same time as an end of reading a final line of a frame that is being read by the imaging device”, ON time of the light source that irradiates white light is shorter than one frame period as illustrated in B in FIG. 8.

Thus, similarly to the case in which the first control is performed in the timing according to the first example indicated in (A) described above, as the ON time of the light source that irradiates white light is set to be shorter than one frame period, the light-source control unit 170 increases one of or both of a light amount of the light source that irradiates white light and a gain of a signal indicating a medical captured image, accordingly.

(C) Third Example of Predetermined Timing

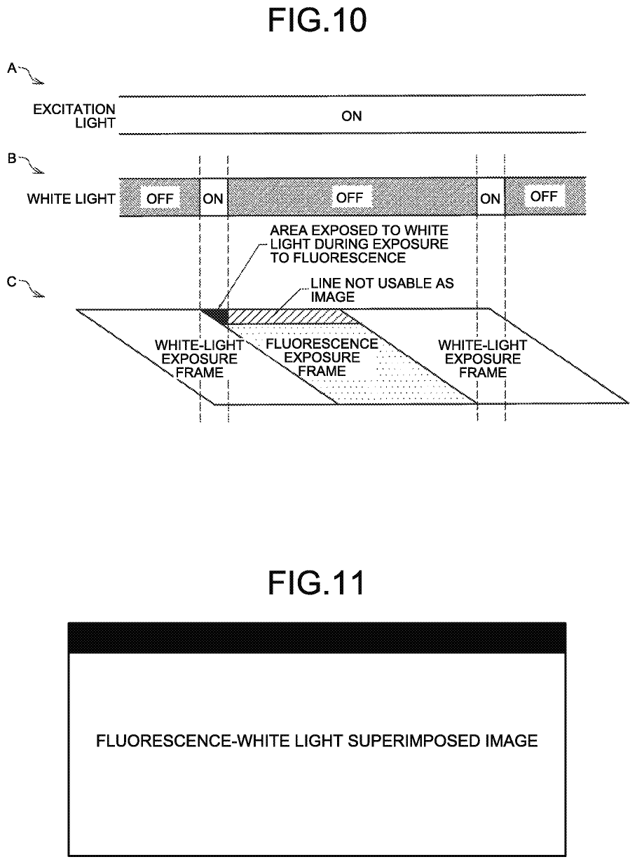

The predetermined timing in which the first control is performed is a timing in which the light source that irradiates white light starts to be ON at the same time as a start of reading a front line of a frame to be read next. FIG. 10 is an explanatory diagram illustrating a third example of a timing of the first control according to the light-source control method according to the present embodiment. A in FIG. 10 illustrates an example of an ON state of the light source that irradiates excitation light, and illustrates an example in which excitation light is irradiated all the time. Note that the medical observation apparatus 100 may turn off the light source that irradiates excitation light during when the light source that irradiates white light is ON. B in FIG. 10 illustrates an example of an ON state of the light source that irradiates white light, and illustrates the third example of a timing of the first control. C in FIG. 10 illustrates one example of a frame that is being read by the imaging device. FIG. 11 is an explanatory diagram illustrating an example of a medical captured image that is obtained when the first control is performed in the timing according to the third example in FIG. 10. The medical captured image illustrated in FIG. 11 is, similarly to FIG. 7, the “example in which a fluorescence captured image obtained by the fluorescence observation and a white-light captured image obtained by the white light observation are synthesized”.

When the first control is performed in the “timing in which the light source that irradiates white light starts to be ON at the same time as a start of reading a front line of a frame to be read next”, time of being exposed to white light is present in a fluorescence exposure frame as it is filled in with black in C in FIG. 10.

Thus, in the medical observation apparatus 100, similarly to the case in which the first control is performed in the timing according to the first example indicated in (A) described above, a portion that is not usable as a fluorescence captured image in a medical observation image (a line portion overlapping the black portion in C in FIG. 10) is masked, not to show the portion on the display screen of the display device 200. A portion in black in FIG. 11 is an example of a masked portion with respect to the medical observation image.

Even when a masked portion is present in a medical observation image as illustrated in FIG. 11, the operator can display a medical captured image in which a range of an observation target necessary for performing procedures is imaged, on the display screen of the display device 200 by one of or both of a zoom operation in the imaging device, and an adjustment of an observation view range by an operation of the arm 104. Therefore, even when a masked portion is present in a medical observation image as illustrated in FIG. 11, there is a low possibility that medical treatment by a medical staff is interfered.

Moreover, when the first control is performed in the “timing in which the light source that irradiates white light starts to be ON at the same time as a start of reading a front line of a frame to be read next”, ON time of the light source that irradiates white light is shorter than one frame period as illustrated in B in FIG. 10.

Thus, similarly to the case in which the first control is performed in the timing according to the first example indicated in (A) described above, as the ON time of the light source that irradiates white light is set to be shorter than one frame period, the light-source control unit 170 increases one of or both of a light amount of the light source that irradiates white light and a gain of a signal indicating a medical captured image, accordingly.

For example, the light-source control unit 170 performs the first control and the second control alternately, and repeats the first control in the timings indicated in (A) to (C) described above. An example of an imaging operation that is achieved by performing the first control and the second control will be described later.

The control according to the light-source control method according to the present embodiment is not limited to the first control and the second control. For example, the light-source control unit 170 may further perform a third control in which the white-light-emitting light-source 166 and the excitation-light-emitting light source 164 are not to be turned on. By performing the third control, a period in which imaging is not performed (so-called blanking period) can be provided.

Referring to FIG. 5 again, an example of a functional configuration of the medical observation apparatus 100 will be described. The processing unit 156 includes, for example, a developing unit 172, an image processing unit 174, and an image formatting unit 176, and processes a medical captured image. The processing unit 156 is constituted of, for example, the processor described above (not shown). Note that the processor (not shown) constituting the processing unit 156 and the processor (not shown) constituting the control unit 154 may be the same processor, or may be different processors.

The developing unit 172 performs RAW development with respect to a signal indicating a medical captured image that is transferred from the imaging unit 150, and develops the medical captured image. The developing unit 172 performs development processing based on the frame signal transferred from the frame-signal generating unit 168. Note that a processing method for the RAW development of an image in the developing unit 172 is not particularly limited.

The image processing unit 174 processes the white-light captured image and the fluorescence captured image that are developed by the developing unit 172. Examples of processing by the image processing unit 174 includes synthesizing processing to synthesize the white-light captured image and the fluorescence captured image. As the synthesizing processing, for example, alpha blending can be considered, but the processing method for synthesizing images in the image processing unit 174 is not particularly limited.

Note that the processing in the image processing unit 174 is not limited to the synthesizing processing.

For example, the image processing unit 174 may perform any image processing, such as resolution conversion processing, processing relating to picture in picture (PIP), and processing relating to picture out picture (POP). Moreover, the processing in the image processing unit 174 may be fixed processing determined in advance, or may be changeable based on an operation by a user.

Furthermore, when the image processing unit 174 has an amplifier, the image processing unit 174 can perform processing of changing a gain of a signal indicating a medical captured image.

In the following, a case in which the processing in the image processing unit 174 is the synthesizing processing will be described as an example.

The image formatting unit 176 converts a signal indicating an image processed by the image processing unit 174 to a predetermined image format. The predetermined image format may be an image format set in advance, or may be changeable based on an operation by a user. The image format according to the present embodiment is not particularly limited. An image signal converted into the predetermined image format by the image formatting unit 176 is transmitted to the display device 200 by the communication device (not shown) included in the medical observation apparatus 100, or by an external communication device out of the medical observation apparatus 100.

The processing unit 156 includes, for example, the developing unit 172, the image processing unit 174, and the image formatting unit 176 as illustrated in FIG. 5. Note that a functional configuration of the processing unit 156 illustrated in FIG. 5 is an example, and the functional configuration of the processing unit 156 is not limited to the example illustrated in FIG. 5.

Next, processing related to the light-source control method according to the present embodiment in the processing unit 156 will be described. The processing unit 156 performs, for example, processing indicated in (a) to (d) described below as the processing related to the light-source control method according to the present embodiment. As described above, the processing in the processing unit 156 is not limited to the example described below.

(a) First Example of Processing Related to Light-Source Control Method in Processing Unit 156

The processing unit 156 synthesizes a first medical captured image that is imaged when at least white light is irradiated to an observation target, and a second medical captured image that is imaged when only excitation light is irradiated to the observation target. Note that when at least white light is irradiated to an observation target includes when only white light is irradiated to the observation target, and when both white light and excitation light are irradiated to the observation target.

As described above, the white light has a larger light amount than fluorescence. Therefore, the first medical captured image imaged when at least white light is irradiated to the observation target corresponds to the white-light captured image.

On the other hand, the second medical captured image imaged when only excitation light is irradiated to the observation target corresponds to the fluorescence captured image.

Therefore, by performing the processing according to the first example in the processing unit 156, for example, images as illustrated in FIG. 7, FIG. 9, and FIG. 11 are obtained.

(b) Second Example of Processing Related to Light-Source Control Method in Processing Unit 156

The processing unit 156 varies a gain of a signal indicating a medical captured image between when at least the first control is performed and when only the second control is performed. Varying the gain of a signal indicating a medical captured image can be achieved, for example, by changing a value of a register of the amplifier provided in the processing unit 156.

When at least the first control is performed is when at least white light is irradiated to an observation target. That is, the signal indicating the medical captured image obtained when at least the first control is performed corresponds to a signal indicating a white-light captured image.

On the other hand, when only the second control is performed is when only excitation light is irradiated to an observation target. That is, the signal indicating the medical captured image obtained when only the second control is performed corresponds to a signal indicating a fluorescence captured image.

As described above, the white light has a larger light amount than fluorescence. Therefore, the processing unit 156 increases a gain of the signal indicating the medical captured image when only the second control is performed (that is, when reading a fluorescence exposure frame), to be larger than that when at least the first control is performed.

Note that when an amplifier to change a gain of a signal indicating a medical captured image is provided only in the imaging unit 150, the processing unit 156 does not perform the processing according to the second example.

(c) Third Example of Processing Related to Light-Source Control Method in Processing Unit 156

The processing unit 156 changes the processing between when at least the first control is performed and when only the second control is performed. As described above, a medical captured image obtained when at least the first control is performed corresponds to the white-light captured image, and a medical captured image obtained when only the second control is performed corresponds to the fluorescence captured image.

The processing unit 156 changes the processing of RAW development that is performed when at least the first control is performed and the processing of RAW development that is performed when only the second control is performed.

It is needless to say that the example of changing the processing according to the third example is not limited to the example described above.

(d) Fourth Example of Processing Related to Light-Source Control Method in Processing Unit 156

The processing unit 156 may perform two or more kinds of processing out of the processing according to the first example indicated in (a) to the processing according to the third example indicated in (c) in combination.

The medical observation apparatus 100 has, for example, a functional configuration illustrated in FIG. 5.

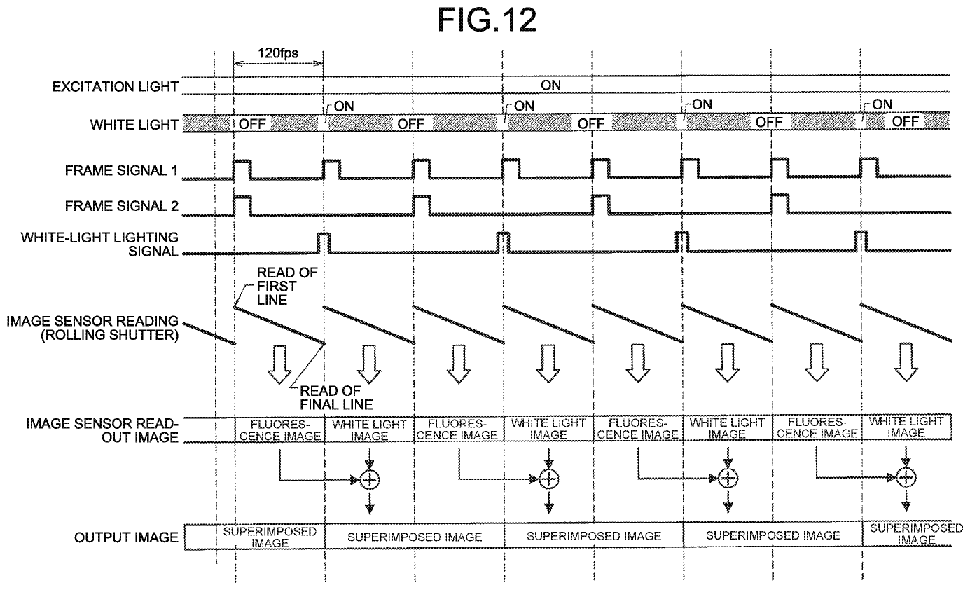

Next, an example of an imaging operation in the medical observation apparatus 100 that has the functional configuration illustrated in FIG. 5 will be described. FIG. 12 is an explanatory diagram illustrating an example of an imaging operation achieved by the first control and the second control according to the light source control method being performed in the medical observation apparatus illustrated in FIG. 5. FIG. 12 illustrates an example in which a basic rate is 120 [fps]. It is needless to say that the example of the imaging operation in the medical observation apparatus 100 is not limited to the example illustrated in FIG. 12.

A white-light lighting signal generated by the frame-signal generating unit 168 is provided to the light-source control unit 170, and the light-source control unit 170 keeps the excitation-light-emitting light source 164 ON all the time, and turns on the white-light-emitting light source 166 according to the white-light lighting signal. Excitation light and white light are irradiated to an observation target, such as a part to be treated, and reflected light and excited fluorescence are irradiated to the excitation-light cut filter 160. In the excitation-light cut filter 160, light in a wavelength band of the excitation light is cut off, and light other than that is irradiated to the image sensor 162. In the image sensor 162, the light that has entered therein is subjected to photoelectric conversion, and in the imaging unit 150, the photoelectric-converted signals are sequentially read from a front line by rolling shutter. The image sensor 162 identifies a frame read-out timing based on a frame signal 1. As described above, fluorescence has a small light amount. Therefore, when the imaging unit 150 is equipped with an amplifier, the imaging unit 150 amplifies the signal, at the time of reading out a fluorescence exposure frame, and then reads it out.

The developing unit 172 identifies a frame read-out timing based on the frame signal 1, and recognizes timings of a fluorescence exposure frame and a white-light exposure frame based on a frame signal 2, to develop a fluorescence captured image and a white-light captured image, respectively. Furthermore, the developing unit 172 masks a line not usable as an image in monochrome, as illustrated in FIG. 7, FIG. 9, and FIG. 11. Note that masking of a line not usable as an image may be performed by another component, as described above.

The image processing unit 174 synthesizes, for example, a fluorescence captured image and a white-light captured image, to generate a fluorescence-white-light superimposed image as illustrated in FIG. 11. Note that the processing in the image processing unit 174 is not limited to synthesizing processing, as described above.

The image formatting unit 176 outputs a signal formatted as, for example, a video signal. It is needless to say that the processing by the image formatting unit 176 is not limited to formatting into a video signal.

The functional configuration of the medical observation apparatus according to the present embodiment is not limited to the configuration illustrated in FIG. 5. For example, the light source unit 152 illustrated in

FIG. 5 may be an external light source provided outside the medical observation apparatus, and the processing unit 156 illustrated in FIG. 5 may be an external processing device provided outside the medical observation apparatus. Even when one or both of the light source unit 152 and the processing unit 156 are absent in the medical observation apparatus according to the present embodiment, the medical observation apparatus according to the present embodiment can perform the processing related to the light-source control method according to the present embodiment, and can produce a similar effect to that of the medical observation apparatus 100 illustrated in FIG. 5.

Moreover, the medical observation apparatus according to the present embodiment may further include a communication unit (not shown) that communicates with an external device, such as the display device 200, in wireless communication or wired communication. The communication unit is constituted of, for example, the communication device (not shown) described above. Communications in the communication unit is controlled by, for example, the control unit 154.

Moreover, the functional configuration that enables to perform the processing related to the light-source control method in the medical observation apparatus according to the present embodiment is not limited to the configuration illustrated in FIG. 5. For example, the configuration can take a functional configuration according to how the processing related to the light-source control method is divided.

Furthermore, when the medical observation apparatus according to the present embodiment has the configuration illustrated in FIG. 1, the medical observation apparatus according to the present embodiment includes an arm unit (not shown) constituted of the arm 104. The arm 104 constituting the arm unit supports the imaging device 106 constituting the imaging unit 150.

Moreover, the medical observation apparatus according to the present embodiment may further include a filter that cuts off light of a wavelength corresponding to fluorescence based on excitation light, arranged in an irradiation path of the white-light-emitting light source 166. When a wavelength of fluorescence emitted from an observation target as a result of irradiation of excitation light to the observation target is included in a wavelength band of white light, a fluorescent wavelength is included in reflected light of white light irradiated from the white-light-emitting light source 166, and a wavelength of fluorescence originated in white light can be photoelectric-converted by the image sensor 162 constituting the imaging unit 150. By arranging the filter that cuts off light of a wavelength corresponding to fluorescence in the irradiation path of the white-light-emitting light source 166, a fluorescent wavelength can be prevented to be included in reflected light of white light that is irradiated from the white-light-emitting light source 166. Accordingly, by arranging the filter that cuts off light of a wavelength corresponding to fluorescence in the irradiation path of the white-light-emitting light source 166, for example, it is possible to control fluorescence in an image obtained by synthesizing a fluorescence captured image and a white-light captured image to be only fluorescence originated from excitation light. The filter that cuts off light of a wavelength corresponding to fluorescence may be a detachable filter, or a fixed filter.

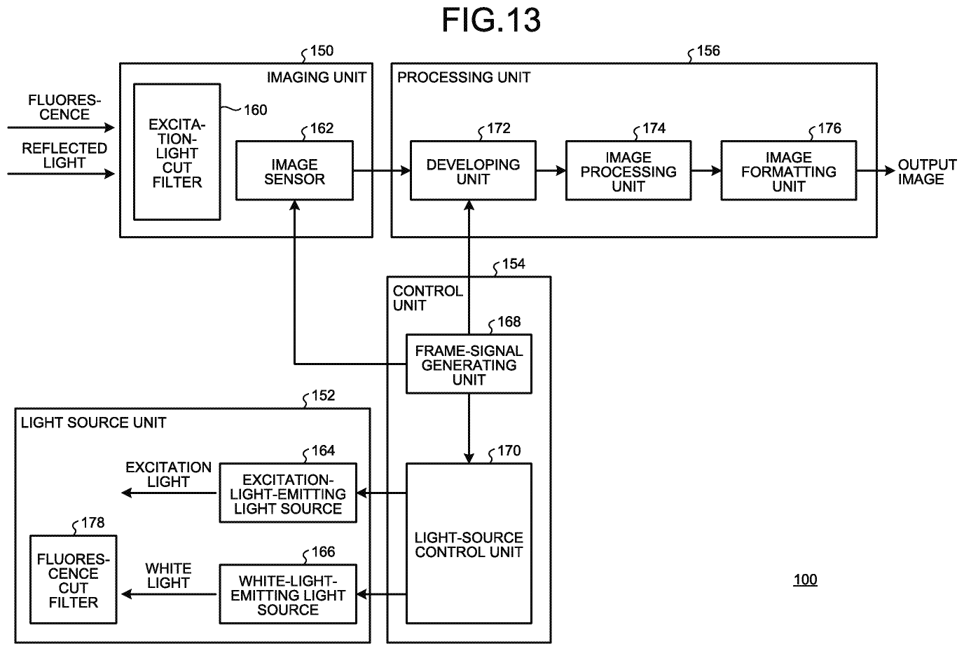

FIG. 13 is a functional block diagram illustrating another example of the configuration of the medical observation apparatus according to the present embodiment. The medical observation apparatus illustrated in FIG. 13 has basically the same configuration as the medical observation apparatus 100 illustrated in FIG. 5, but differs in a point in which a fluorescence cut filter 178 (filter cutting off light of a wavelength corresponding to fluorescence) is arranged in an irradiation path of the white-light-emitting light source 166. Therefore, the medical observation apparatus illustrated in FIG. 13 basically produces an effect similar to that of the medical observation apparatus 100 illustrated in FIG. 5, and can prevent a fluorescent wavelength from being included in reflected light of white light that is irradiated from the white-light-emitting light source 166.

The medical observation apparatus illustrated in FIG. 13 can take a modification similar to that of the medical observation apparatus 100 illustrated in FIG. 5.

[3] One Example of Effect Produced by Using Light-Source Control Method According to Present Embodiment

By applying the light-source control method according to the present embodiment, for example an effect described below is produced. It is needless to say that an effect produced by applying the light-source control method according to the present embodiment is not limited to the example described below.

By the configuration and the operation of the medical observation apparatus described above, for example, a “superimposed image of a fluorescence captured image of 60 fps and a white-light captured image is obtained by time division imaging by one image sensor, and white light can be made to be flashing at 60 [Hz]”. Therefore, by the configuration and the operations of the medical observation apparatus described above, reduction in frame rate can be prevented, and discomfort caused by flicker can be reduced, thereby improving the usability for a user of the medical observation apparatus.

Program According Present Embodiment

By executing a program (for example, a program enabling to perform processing the according to the light-source control method according to the present embodiment) that causes a computer system to function as the medical observation apparatus according to the present embodiment, by a processor or the like, the usability for a user of the medical observation apparatus can be improved. As the computer system according to the present embodiment, a single computer or a multiple computers may be considered. By the computer system according to the present embodiment, a series of processing related to the light-source control method according to the present embodiment is performed.

Furthermore, with the program that causes the computer system to function as the medical observation apparatus according to the present embodiment being executed by a processor or the like in the computer system, the effect produced by display implemented by processing related to the light-source control method according to the present embodiment described above can be produced.

As above, the exemplary embodiments of the present disclosure have been described in detail with reference to the accompanying drawings, but a technical scope of the present disclosure is not limited to the examples. It is obvious that those having general knowledge in the technical field of the present disclosure can think of various alterations or modifications within a range of technical thought described in claims, and is understood that these, of course, are included in the technical scope of the present disclosure also.

For example, in the above, it is described that a program (computer program) to cause a computer system to function as the medical observation apparatus according to the present embodiment is provided, but the present embodiment can also provide a recording medium that stores the program along therewith.

The configuration described above indicates an example of the present embodiment and, of course, is included in the technical scope of the present disclosure.

Moreover, the effects described in the present application are only for explanation and exemplification, and are not limited. That is, the technique according to the present disclosure can produce other effects that are apparent from description of the present application to those skilled in the art instead of the above effects.

The image sensor may be inserted into an inside of a body of a patient, and images the inside the body as the observation target.

According to the present disclosure, it is possible to improve usability for a user of a medical observation apparatus.

The above effect is not limited, but together with or instead of the above effect, any effect described in the application, or other effects that can be thought of from the present application may be produced.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical observation apparatus comprising:
an image sensor configured to image an observation target and generate a medical captured image by sequential reading; and
a controller configured to control lighting of light sources configured to irradiate light to the observation target, wherein
the controller is configured to perform a first control including a control of lighting a light source configured to irradiate white light, and a second control including a control of lighting a light source configured to irradiate excitation light, and
the first control is performed in a period that spans over a boundary of successive frames.

2. The medical observation apparatus according to claim 1, wherein the controller is configured to perform:
the first control in a shorter period than one frame period; and
the second control in a period longer than the period in which the first control is performed.

3. The medical observation apparatus according to claim 1, wherein the controller is configured to:
repeat the first control; and
perform the second control in a period in which at least the first control is not performed.

4. The medical observation apparatus according to claim 3, wherein the controller is configured to perform the second control in a period in which the first control is performed, and in a period in which the first control is not performed.

5. The medical observation apparatus according to claim 3, wherein the controller is configured to perform the second control in a period in which the first control is not performed, and is configured not to perform the second control in a period in which the first control is performed.

6. The medical observation apparatus according to claim 3, wherein the controller is configured to perform the first control in a predetermined timing that has been set.

7. The medical observation apparatus according to claim 6, wherein the predetermined timing is a timing in which lighting of the light source configured to irradiate the white light ends at the same time as an end of reading a final line of a frame that is being read by the image sensor.

8. The medical observation apparatus according to claim 6, wherein the predetermined timing is a timing in which the light source configured to irradiate the white light starts to be ON at the same time as a start of reading a front line of a frame to be read next.

9. The medical observation apparatus according to claim 6, wherein the predetermined timing is a timing in which lighting of the light source configured to irradiate the white light spans over an end of reading a final line of a frame that is being read in the imaging device and a start of reading a front line of a frame to be read next.

10. The medical observation apparatus according to claim 3, wherein the controller is configured to perform the first control every two frame period.

11. The medical observation apparatus according to claim 1, wherein the controller is further configured to perform a third control in which the light source configured to irradiate the white light and the light source configured to irradiate the excitation light are not turned on.

12. The medical observation apparatus according to claim 1, further comprising a processor configured to processe the medical captured image.

13. The medical observation apparatus according to claim 12, wherein the processor is configured to vary a gain of a signal indicating the medical captured image between when at least the first control is performed and when only the second control is performed.

14. The medical observation apparatus according to claim 12, wherein the processor is configured to change processing between when at least the first control is performed and when only the second control is performed.

15. The medical observation apparatus according to claim 12, wherein the processor is configured to synthesize a first medical captured image that is imaged when at least the white light is irradiated to the observation target, and a second medical captured image that is imaged when only the excitation light is irradiated to the observation target.

16. The medical observation apparatus according to claim 1, further comprising a filter configured to cut off light of a wavelength corresponding to the excitation light from incident light to the image sensor.

17. The medical observation apparatus according to claim 1, further comprising a light source unit including a light source configured to irradiate the white light and a light source configured to irradiate the excitation light.

18. The medical observation apparatus according to claim 17, wherein the light source unit further includes a filter configured to pass the irradiated white light, and cut off light of a wavelength corresponding to fluorescence originated from the excitation light.

19. The medical observation apparatus according to claim 1, further comprising an arm including a plurality of links connected with one another by a joint portion, wherein
the image sensor is supported by the arm.
A medical observation system comprising:
a medical observation apparatus configured to image an observation target, and process a generated medical captured image; and
a display configured to display the processed medical captured image on a display screen, wherein
the medical observation apparatus includes
an image sensor configured to image the observation target and generate the medical captured image by sequential reading;
a controller configured to control lighting of light sources configured to irradiate light to the observation target; and
a processor configured to process the medical captured image, wherein
the controller is configured to perform a first control including a control of lighting a light source configured to irradiate white light, and a second control including a control of lighting a light source configured to irradiate excitation light, and
the first control is performed in a period that spans over a boundary of successive frames.
